(19) United States
(12) Patent Application Publication
(10) Pub. No.: US 2009/0202647 A1
(43) Pub. Date: Aug. 13, 2009

(54) SOLID FORM OF RACEMIC ROTIGOTINE

(76) Inventors: Mayur Devjibhai Khunt, Junagadh (IN); Nilesh Sudhir Patil, Maharashtra (IN); Nitin Sharadchandra Pradhan, Maharashtra (IN); Jon Valgeirsson, Hafnarfjordur (IS)

Correspondence Address: CANTOR COLBURN, LLP, 20 Church Street, 22nd Floor, Hartford, CT, 06103, US

(21) Appl. No.: 12/369,031

(22) Filed: Feb. 11, 2009

(30) Foreign Application Priority Data
Feb. 11, 2008 (IN) 350/CHE/2008

(51) Int. Cl. A61K 9/14; C07D 333/20; A61K 31/381

(52) U.S. Cl. 424/489; 549/75; 514/438

(57) ABSTRACT

Disclosed herein are solid state forms, amorphous and crystalline forms, of racemic rotigotine having high purity, adequate stability, good flowability and good dissolution properties, a process for preparation, and pharmaceutical compositions comprising amorphous racemic rotigotine.

### CROSS REFERENCE TO RELATED APPLICATION

This application claims the benefit of priority to Indian provisional application No. 350/CHE/2008, filed on Feb. 11, 2008, which is incorporated herein by reference.

### FIELD OF THE DISCLOSURE

The present disclosure relates to solid state forms of racemic rotigotine, processes for the preparation, pharmaceutical compositions, and method of treating thereof.

### BACKGROUND

U.S. Pat. No. 4,564,628 discloses a variety of substituted 2-aminotetralin derivatives, processes for their preparation, pharmaceutical compositions and method of use thereof. These compounds are useful as dopamine agonists and, in particular, dopamine D-2 receptor agonists for the treatment of disorders of the central nervous, cardiovascular and endocrine systems such as Parkinson's disease and related disorders, hypertension and hyper-prolactinemia. In particular, the compounds are useful in the treatment of glaucoma in mammals. Among them, racemic rotigotine, 5-hydroxy-2-[N-n-propyl-N-2-(2-thienyl)ethylamino]tetralin, is a non-ergoline D₃/D₂/D₁ dopamine agonist for the treatment of Parkinson's disease. Rotigotine is represented by the following structural formula:

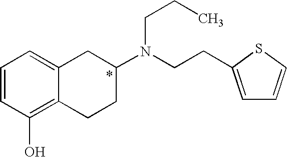

The structural formula of rotigotine contains one chiral center (the asterisk designates the chiral center) and therefore exist as two optical isomers. Various processes for the preparation of 5-hydroxy-2-[N-n-propyl-N-2-(2-thienyl)ethylamino]tetralin and related compounds are disclosed in U.S. Pat. No. 4,564,628, European Patent No. 168505, U.S. Pat. No. 4,657,925, U.S. Pat. No. 4,968,837 and U.S. Pat. No. 6,372,920.

According to the U.S. Pat. No. 4,564,628, 5-hydroxy-2-[N-n-propyl-N-2-(2-thienyl)ethylamino]tetralin (hereinafter referred to as the “racemic rotigotine free base”) is isolated as a hydrochloride salt according to conventional techniques. According to the embodiment exemplified in Example 2, the racemic rotigotine hydrochloride was prepared by dissolving 5-methoxy-2-[N-n-propyl-N-2-(2-thienyl)ethylamino]tetralin in dichloromethane, cooling the solution to −30° C., followed by addition of BBr₃ and stirring for 2 hours to form a reaction mass. To the reaction mass methanol was added to form a clear solution, followed by extractions with saturated solution of NaHCO₃ and water. The resulting organic layer was dried over anhydrous MgSO₄, reduced to dryness, followed by conversion to a hydrochloride salt.

U.S. Pat. No. 4,657,925 describes both the (−)-enantiomer and (+)-enantiomer for rotigotine, of which the levo (−) isomer is reported to be more active when used in therapeutic treatment.

U.S. Pat. No. 6,372,920 describes a process for preparing optically active and racemic nitrogen-substituted 2-aminotetralins including rotigotine.

U.S. Patent Application No. 2005/0175678 A1 describes a matrix suitable for transdermal administration of the levo (−) isomer of rotigotine [(−)-5,6,7,8-tetrahydro-6-[propyl[2-(2-thienyl)ethyl]amino]-1-naphthol] that is free of solvents and dispersants. The matrix comprises at least one matrix polymer and rotigotine base in a concentration above the solubility limit of the matrix polymer for rotigotine, wherein the portion of the rotigotine not dissolved in the matrix polymer is dispersed in the matrix polymer as amorphous particles with a maximum mean diameter of 30 μm.

The references cited above do not describe solid forms of racemic rotigotine free base.

### SUMMARY

The present inventors have now surprisingly and unexpectedly found solid state forms of racemic rotigotine, including amorphous and crystalline forms, with high purity, adequate stability, good flowability and good dissolution properties.

In one aspect, racemic rotigotine free base in a solid state is provided. In another aspect, racemic rotigotine free base in a crystalline form is provided. In yet another aspect, racemic rotigotine free base in an amorphous form is provided. In another aspect, the solid state forms of racemic rotigotine free base exists in an anhydrous and/or solvent-free form or as a hydrate and/or a solvate form.

In another aspect, encompassed herein is a process for preparing the highly pure and stable solid state forms of racemic rotigotine including amorphous and crystalline forms.

In another aspect, provided herein are highly pure solid state forms of racemic rotigotine free base and use thereof for the preparation of levo (−)-isomer of rotigotine i.e., (−)-(S)-5-Hydroxy-2-[N-n-propyl-N-2-(2-thienyl)ethylamino]tetralin.

In another aspect, provided is a pharmaceutical composition comprising solid state forms of racemic rotigotine and one or more pharmaceutically acceptable excipients.

In still another aspect, provided is a pharmaceutical composition comprising solid state form of racemic rotigotine made by the process disclosed herein, and one or more pharmaceutically acceptable excipients.

In still further aspect, encompassed is a process for preparing a pharmaceutical formulation comprising combining solid state form of racemic rotigotine with one or more pharmaceutically acceptable excipients.

### BRIEF DESCRIPTION OF THE DRAWINGS

FIG. 1 is a characteristic powder X-ray diffraction (XRD) pattern of amorphous form of racemic rotigotine.

FIG. 2 is a characteristic Infrared (IR) spectrum of amorphous form of racemic rotigotine.

FIG. 3 is a characteristic powder X-ray diffraction (XRD) pattern of crystalline form of racemic rotigotine.

### DETAILED DESCRIPTION

Disclosed herein is the unexpected discovery that racemic rotigotine free base can be obtained as a solid. Since the solid form of racemic rotigotine free base is obtained with high purity, the solid can be used to obtain pharmaceutically acceptable salts of racemic rotigotine free base in high purity. It has been found that purification of crude racemic rotigotine free base is practically advantageous when compared with the purification of a salt form.

In the formulation of drug compositions, it is important for the active pharmaceutical ingredient to be in a form in which it can be conveniently handled and processed. Convenient handling is important not only from the point of view of obtaining a commercially viable manufacturing process, but also from the point of view of subsequent manufacture of pharmaceutical formulations (e.g., an ointment, a gel, a cream, a paste, a spray, a film, a plaster, a patch, a poultice, a cataplasm or an iontophoretic device) comprising the active pharmaceutical ingredient.

Chemical stability, solid state stability, and “shelf life” of the active pharmaceutical ingredient are important properties for a pharmaceutically active compound. The active pharmaceutical ingredient, and compositions containing it, should be capable of being effectively stored over appreciable periods of time, without exhibiting a significant change in the physico-chemical characteristics of the active pharmaceutical ingredient, e.g., its chemical composition, density, hygroscopicity and solubility. Thus, in the manufacture of commercially viable and pharmaceutically acceptable drug compositions, it is important, wherever possible, to provide the active pharmaceutical ingredient in a stable form.

New solid forms of a pharmaceutical agent can further the development of formulations for the treatment of illnesses. For instance, solid forms of a compound are known in the pharmaceutical arts to affect, for example, the solubility, dissolution rate, bioavailability, chemical and physical stability, flowability, tractability, and compressibility of the compound, as well as the safety and efficacy of drug products based on the compound.

The discovery of novel solid forms, including amorphous forms and crystal forms, of pharmaceutically useful compounds provides a new opportunity to improve the performance characteristics of a pharmaceutical product. It also adds value to the material that a formulation scientist can use the same for designing, for example, a pharmaceutical dosage form of a drug with a targeted release profile or other desired characteristic. Novel solid forms of racemic rotigotine have now been discovered.

It has been disclosed in the art that the amorphous forms of a number of drugs exhibit superior dissolution characteristics and in some cases different bioavailability patterns compared to crystalline forms [Konno T., Chem. Pharm. Bull., 38, 2003 (1990)]. For some therapeutic indications one bioavailability pattern may be favored over another.

According to one aspect, there is provided solid state forms of racemic rotigotine free base. In another aspect, racemic rotigotine free base in a crystalline form is provided. In yet another aspect, racemic rotigotine free base in an amorphous form is provided. In another aspect, the solid state forms of racemic rotigotine free base exist in an anhydrous and/or solvent-free form or as a hydrate and/or a solvate forms.

In another embodiment, the solid state forms of racemic rotigotine free base are useful intermediates in the preparation of levo (−)-isomer of rotigotine, i.e., (−)-(S)-5-Hydroxy-2-[N-n-propyl-N-2-(2-thienyl)ethylamino]tetralin or a pharmaceutically acceptable salt thereof in high purity. The solid state forms of racemic rotigotine free base have good flow properties and are stable at room temperature, enhanced temperature and at relative high humidities and in aqueous media, and so, the solid state forms are suitable for formulating racemic rotigotine.

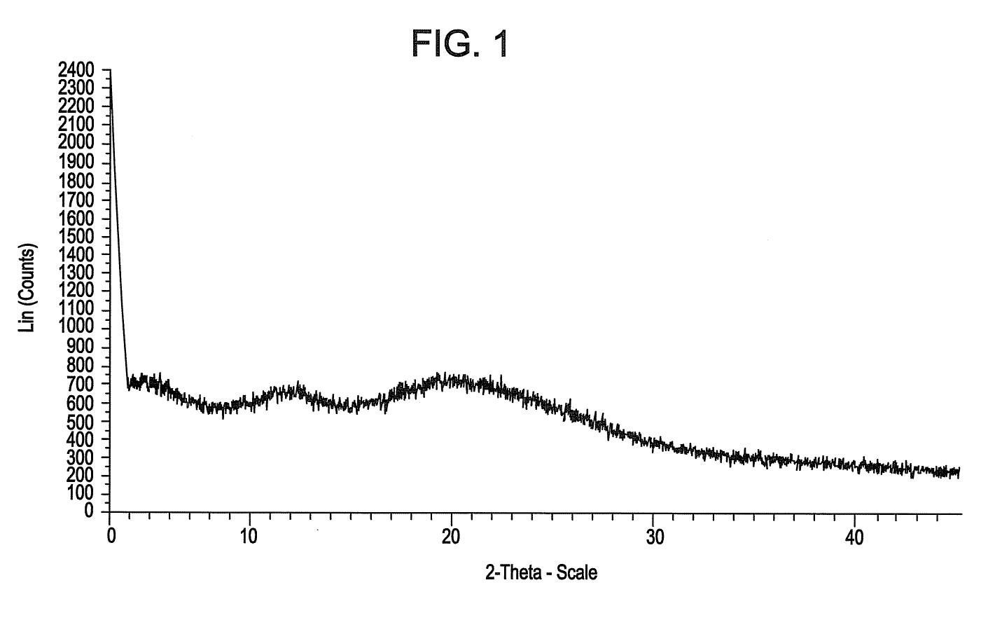

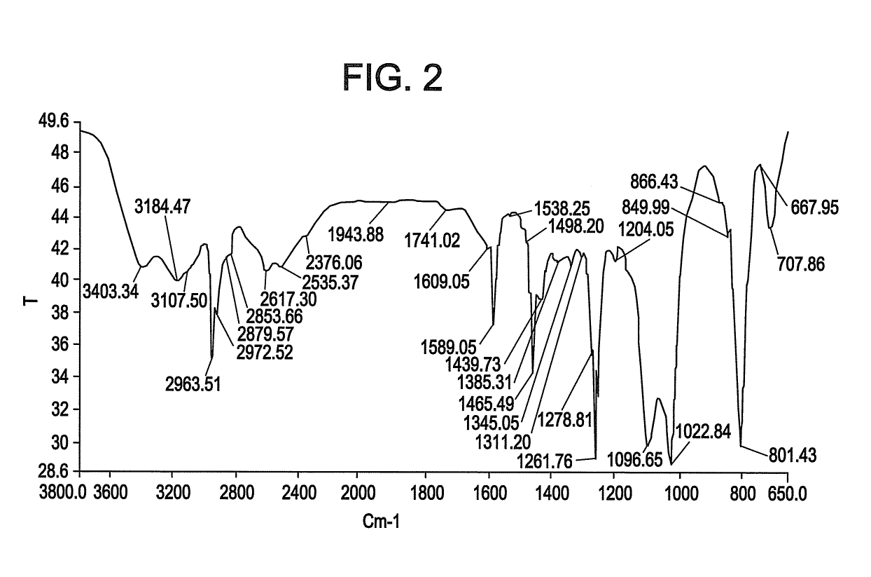

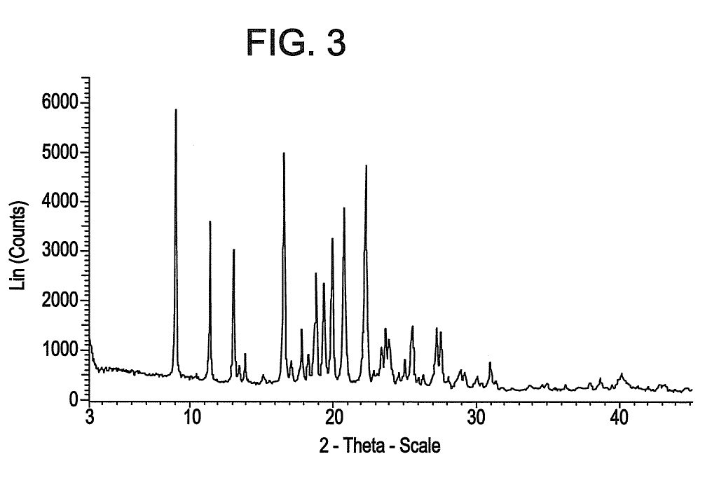

The solid state form of racemic rotigotine free base is characterized by data including:
i) a powder X-ray diffraction pattern substantially in accordance with FIG. 1;
ii) an IR spectrum substantially in accordance with FIG. 2;
iii) a powder X-ray diffraction pattern substantially in accordance with FIG. 3;
iv) a powder X-ray diffraction pattern having peaks at about 9.11, 11.47, 13.11, 16.63, 18.86, 19.41, 19.97, 20.82 and 22.32±0.2 degrees 2-theta substantially as depicted in FIG. 3; and/or
v) a powder X-ray diffraction pattern having peaks at about 13.92, 17.83, 23.70, 23.94, 25.49, 27.15 and 27.46±0.2 degrees 2-theta substantially as depicted in FIG. 3.

According to another aspect, there is provided a stable and substantially pure amorphous form of racemic rotigotine free base.

The amorphous form of racemic rotigotine is characterized by a powder X-ray diffraction pattern substantially in accordance with FIG. 1. The X-ray powder diffraction pattern shows no peaks, thus demonstrating the amorphous nature of the product.

The amorphous form of racemic rotigotine is further characterized by a Fourier transform Infrared (FT-IR) spectrum substantially in accordance with FIG. 2.

The term “substantially pure amorphous form of racemic rotigotine” refers to the amorphous form of racemic rotigotine having purity greater than about 98%, specifically greater than about 99%, more specifically greater than about 99.5%, and still more specifically greater than about 99.9% (measured by HPLC).

According to another aspect, there is provided a stable and substantially pure crystalline form of racemic rotigotine free base.

The crystalline form of racemic rotigotine free base is characterized by data including:
i) a powder X-ray diffraction pattern substantially in accordance with FIG. 3;
ii) a powder X-ray diffraction pattern having peaks at about 9.11, 11.47, 13.11, 16.63, 18.86, 19.41, 19.97, 20.82 and 22.32±0.2 degrees 2-theta substantially as depicted in FIG. 3; and/or
iii) a powder X-ray diffraction pattern having peaks at about 13.92, 17.83, 23.70, 23.94, 25.49, 27.15 and 27.46±0.2 degrees 2-theta substantially as depicted in FIG. 3.

The term “substantially pure crystalline form of racemic rotigotine” refers to the crystalline form of racemic rotigotine having purity greater than about 98%, specifically greater than about 99%, more specifically greater than about 99.5%, and still more specifically greater than about 99.9% (measured by HPLC).

According to another aspect, a process is provided for preparation of solid state forms of racemic rotigotine free base, comprising:
a) providing a solution or suspension of racemic rotigotine free base in a solvent;
b) optionally, filtering the solution or suspension to remove any extraneous matter; and
c) substantially removing the solvent from the solution or suspension to afford the solid state form of an amorphous form of racemic rotigotine free base; or
d) isolating from the solution or suspension the solid state form of a crystalline form of racemic rotigotine free base.

The term “substantially removing” the solvent or solvent mixture refers to at least 10%, specifically grater than about 50%, more specifically grater than about 90%, still more specifically grater than about 99%, and most specifically essentially complete, removal of the solvent from the solvent solution or the reaction mass containing the solvent or solvent mixture.

The process can produce solid state form of racemic rotigotine free base in substantially pure form.

The term “substantially pure solid state forms of racemic rotigotine free base” refers to the solid state forms of racemic rotigotine free base having purity greater than about 98%, specifically greater than about 99%, more specifically greater than about 99.5% and still more specifically greater than about 99.9% (measured by HPLC).

In another embodiment, the solid state forms of racemic rotigotine are useful intermediates in the preparation of levo (−) isomer of rotigotine i.e., (−)-(S)-5-Hydroxy-2-[N-n-propyl-N-2-(2-thienyl)ethylamino]tetralin or a pharmaceutically acceptable salt thereof in high purity.

In another embodiment, the solid state form of racemic rotigotine has a water content of less than about 5% by weight, specifically less than about 1% by weight, and more specifically less than about 0.5% by weight, and still more specifically is essentially free from water, based on the total weight of the racemic rotigotine free base.

The amorphous racemic rotigotine obtained by the process disclosed herein is stable, consistently reproducible and has good flow properties. Amorphous racemic rotigotine is particularly suitable for bulk preparation and handling, and so, the amorphous racemic rotigotine obtained by the process disclosed herein is suitable for formulating racemic rotigotine.

The crystalline racemic rotigotine obtained by the process disclosed herein is stable, consistently reproducible and has good flow properties. Crystalline racemic rotigotine is particularly suitable for bulk preparation and handling, and so, the amorphous racemic rotigotine obtained by the process disclosed herein is suitable for formulating racemic rotigotine.

Exemplary solvents used in step-(a) include, but are not limited to, alcohols, ketones, hydrocarbons, chlorinated hydrocarbons, esters, and mixtures thereof. The term solvent includes mixtures of solvents.

Specifically, the solvent includes methanol, ethanol, n-propanol, isopropyl alcohol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, methylene chloride, ethylene dichloride, chloroform, carbon tetrachloride, n-pentane, n-hexane, n-heptane, cyclohexane, toluene, xylene, and mixtures thereof; more specifically the solvent is selected from the group consisting of acetone, methanol, ethanol, n-propanol, isopropyl alcohol, n-butanol, isobutanol, tert-butanol, ethyl acetate, isopropyl acetate, methylene chloride, n-hexane, n-heptane, cyclohexane, toluene, and mixtures thereof; and most specifically the solvent is selected from the group consisting of methanol, ethanol, isopropyl alcohol, ethyl acetate, n-hexane, n-heptane, cyclohexane, toluene, and mixtures thereof.

Step-(a) of providing a solution or suspension of racemic rotigotine free base includes dissolving or suspending racemic rotigotine free base in the solvent, or obtaining an existing solution or suspension from a previous processing step.

In one embodiment, the racemic rotigotine free base is dissolved in the solvent at a temperature of about 30° C. to the reflux temperature of the solvent used, more specifically at about 40° C. to about 100° C., and still more specifically at about 50° C. to about 80° C.

In another embodiment, step-(a) of forming a suspension of racemic rotigotine free base includes suspending racemic rotigotine free base in the solvent under stirring at below reflux temperature of the solvent used. Preferably, the suspension is stirred at a temperature of about 30° C. to about 100° C. for at least 15 minutes, and more preferably at about 40° C. to about 80° C. from about 30 minutes to about 5 hours.

As used herein, “reflux temperature” means the temperature at which the solvent or solvent system refluxes or boils at atmospheric pressure.

In an embodiment, the solution or suspension is prepared by reacting 5-Methoxy-2-[N-n-propyl-N-2-(2-thienyl)ethylamino]tetralin with HBr or BBr₃ or with thiourea in the presence of aluminium chloride, under suitable conditions to produce a reaction mass containing racemic rotigotine free base, followed by usual work up such as filtrations, washings, extractions, acid base treatments etc., and dissolving or suspending the resulting racemic rotigotine free base in the suitable solvent or solvent mixture at a temperature of about 30° C. to the reflux temperature of the solvent used, more specifically at about 40° C. to about 100° C., and still more specifically at about 50° C. to about 80° C.

Alternatively, the solution or suspension is prepared by treating an acid addition salt of racemic rotigotine with a base to liberate racemic rotigotine free base and dissolving or suspending the racemic rotigotine free base in a solvent.

In an embodiment, the acid addition salts are derived from a therapeutically acceptable acid such as hydrochloric acid, acetic acid, propionic acid and, more particularly, from a di- or polybasic acid such as phosphoric acid, succinic acid, maleic acid, fumaric acid, citric acid, glutaric acid, citraconic acid, glutaconic acid, tartaric acid, malic acid, and ascorbic acid. A more preferable salt is racemic rotigotine hydrochloride.

The treatment of an acid addition salt with base is carried out in a solvent and the selection of solvent is not critical. A wide variety of solvents such as chlorinated solvents, alcohols, ketones, hydrocarbon solvents, ether solvents etc., can be used.

The base can be inorganic or organic. An exemplary base is an inorganic base selected from alkali metal hydroxides, carbonates and bicarbonates. A preferred alkali metal is sodium or potassium.

The solution or suspension obtained in step-(a) is optionally subjected to carbon treatment or silica gel treatment. The carbon treatment or silica gel treatment is carried out by methods known in the art, for example, by stirring the solution with finely powdered carbon or silica gel at a temperature of below about 70° C. for at least 15 minutes, preferably at a temperature of about 40° C. to about 70° C. for at least 30 minutes; and filtering the resulting mixture through hyflo to obtain a filtrate containing racemic rotigotine free base by removing charcoal or silica gel. Preferably, finely powdered carbon is an active carbon. Preferable mesh size of silica gel is 60-120 mesh.

The solution or suspension obtained in step-(a) or step-(b) is optionally stirred at a temperature of about 30° C. to the reflux temperature of the solvent used for at least 20 minutes, and specifically at a temperature of about 40° C. to the reflux temperature of the solvent used from about 30 minutes to about 4 hours.

Removal of solvent in step-(c) is accomplished, for example, by substantially complete evaporation of the solvent, concentrating the solution or distillation of solvent, under inert atmosphere to obtain amorphous racemic rotigotine free base.

In one embodiment, the solvent is removed by evaporation. Evaporation can be achieved at sub-zero temperatures by the lyophilisation or freeze-drying techniques. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

The distillation process can be performed at atmospheric pressure or reduced pressure. Specifically, the solvent is removed at a pressure of about 760 mm Hg or less, more specifically at about 400 mm Hg or less, still more specifically at about 80 mm Hg or less, and most specifically from about 30 to about 80 mm Hg.

Solvents can also be removed by spray-drying, in which a solution of racemic rotigotine free base is sprayed into the spray drier at the flow rate ranging from 10 to 300 ml/hr, preferably flow rate is 40 to 200 ml/hr. The air inlet temperature to the spray drier used may range from about 30° C. to about 150° C., specifically from about 65° C. to about 110° C. and the outlet air temperature used may range from about 30° C. to about 90° C.

Another suitable method is vertical agitated thin-film drying (or evaporation). Agitated thin film evaporation technology involves separating the volatile component using indirect heat transfer coupled with mechanical agitation of the flowing film under controlled condition. In vertical agitated thin-film drying (or evaporation) (ATFD-V), the starting solution is fed from the top into a cylindrical space between a centered rotary agitator and an outside heating jacket. The rotor rotation agitates the downside-flowing solution while the heating jacket heats it.

The isolation of crystalline form of racemic rotigotine free base in step-(d) is carried out by forcible or spontaneous crystallization.

Spontaneous crystallization refers to crystallization without the help of an external aid, such as seeding, cooling etc., and forcible crystallization refers to crystallization with the help of an external aid.

Forcible crystallization may be initiated by a method known in the art such as cooling, seeding, partial removal of the solvent from the solution, by combining an anti-solvent with the solution or a combination thereof.

In one embodiment, the crystallization is carried out by cooling the solution at a temperature of below 30° C. for at least 30 minutes, specifically at about 0° C. to about 30° C. from about 1 hour to about 20 hours, and more specifically at about 0° C. to about 10° C. from about 2 hours to about 10 hours.

The crystalline form of racemic rotigotine free base obtained in step-(d) may be recovered by conventional techniques known in the art such as filtration, filtration under vacuum, decantation, and centrifugation, or a combination thereof. In one embodiment, the crystalline racemic rotigotine free base is isolated by filtration employing a filtration media of, for example, a silica gel or celite.

The pure solid state forms of racemic rotigotine free base obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying is carried out at atmospheric pressure or reduced pressures, such as below about 200 mm Hg, or below about 50 mm Hg, at temperatures such as about 25° C. to about 70° C. The drying can be carried out for a desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The purity of the solid state forms of racemic rotigotine free base obtained by the process disclosed herein are of greater than about 99%, specifically greater than about 99.5%, more specifically greater than about 99.9%, and most specifically greater than about 99.95% as measured by HPLC. For example, the purities of the amorphous and crystalline racemic rotigotine free base of the present invention can be about 99% to about 99.95%, or about 99.5% to about 99.99%.

Further encompassed herein is the use of solid state forms of racemic rotigotine free base for the manufacture of a pharmaceutical composition.

Preparation of racemic rotigotine free base as solid affords pure racemic rotigotine, which can be resolved using a suitable optically active resolving agent by known methods to produce levo (−) isomer of rotigotine i.e., (−)-(S)-5-Hydroxy-2-[N-n-propyl-N-2-(2-thienyl)ethylamino]tetralin or a pharmaceutically acceptable salt thereof in high purity.

In one embodiment, the solid state forms of racemic rotigotine free base have a D₉₀ particle size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the substantially pure solid state forms of racemic rotigotine free base disclosed herein for use in the pharmaceutical compositions have a 90 volume-percent of the particles (D₉₀) of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of solid state forms of racemic rotigotine free base can be achieved by a mechanical process of reducing the size of particles which includes any one or more of cutting, chipping, crushing, milling, grinding, micronizing, trituration or other particle size reduction methods known in the art, to bring the solid state forms the desired particle size range.

Further encompassed herein is the use of amorphous forms of racemic rotigotine free base for the manufacture of a pharmaceutical composition together with a pharmaceutically acceptable carrier.

Also included is the use of crystalline form of racemic rotigotine free base of for the manufacture of a pharmaceutical composition together with a pharmaceutically acceptable carrier.

According to another aspect, there are provided pharmaceutical compositions comprising amorphous racemic rotigotine free base prepared according to process disclosed herein and one or more pharmaceutically acceptable excipients.

According to another aspect, there is provided a process for preparing a pharmaceutical formulation comprising combining amorphous racemic rotigotine free base prepared according to process disclosed herein, with one or more pharmaceutically acceptable excipients.

According to another aspect, there is provided pharmaceutical compositions comprising crystalline racemic rotigotine free base prepared according to process disclosed herein and one or more pharmaceutically acceptable excipients.

According to another aspect, there is provided a process for preparing a pharmaceutical formulation comprising combining crystalline racemic rotigotine free base prepared according to process disclosed herein, with one or more pharmaceutically acceptable excipients.

There are many methods of application available for administering racemic rotigotine, which the person skilled in the art can select and adapt depending on the need, condition and age of the subject, the required dosage and the desired application interval. As non-limiting examples, the route of administration can be parenteral, transdermal or transmucosal.

In an embodiment, the route of administering the solid state forms of racemic rotigotine free base, including amorphous and crystalline forms, is parenteral, transdermal or transmucosal administration.

In a specific embodiment, the route of administering the solid state forms of racemic rotigotine free base is transdermal administration. The form and pharmaceutical composition in which the compound is administered is adapted for the route of administration and, in the case of transdermal administration, a suitable composition can be, for example, an ointment, a gel, a cream, a paste, a spray, a film, a plaster, a patch, a poultice, a cataplasm or an iontophoretic device.

In a particular embodiment, solid state forms of racemic rotigotine free base may be administered by application to a patient's skin of a patch or plaster having the active substance present in an adhesive polymer matrix, for instance a self-adhesive polysiloxane matrix.

The term “transdermal therapeutic system”, or its abbreviation “TTS”, as used herein refers to a pharmaceutical composition, in a form of one to a plurality of patch or plaster formulations, that contains an active agent, for example amorphous or crystalline form of racemic rotigotine, and that when applied to skin of a subject delivers at least a portion of the active agent into and across the skin, where the active agent accesses the circulatory system of the subject. A transdermal therapeutic system useful herein can be prepared by processes known in the art.

The following examples are provided to enable one skilled in the art to practice the invention and are merely illustrate the process of this invention. However, it is not intended in any way to limit the scope of the present invention.

### EXAMPLES

X-Ray powder diffraction spectra were measured by an X-ray powder diffractometer equipped with a Cu-anode (λ=1.54 Angstrom), X-ray source operated at 40 kV, 40 mA and a Ni filter is used to strip K-beta radiation. Two-theta calibration was performed using an NIST SRM 1976, Corundum standard. The sample was analyzed using the following instrument parameters: measuring range=3-45° 2θ; step width=0.01579; and measuring time per step=0.11 second.

FT-IR spectroscopy was carried out with a Perkin Elmer Spectrum 100 series spectrometer. For the production of the KBr compacts, approximately 2 mg of sample was powdered with 200 mg of KBr. The spectra were recorded in transmission mode ranging from 3800 to 650 cm⁻¹.

### Example 1

### Preparation of Amorphous Form of Racemic Rotigotine

5-Methoxy-2-[N-n-propyl-N-2-(2-thienyl)ethylamino]tetralin (0.5 gm) was added to a round bottom flask followed by the addition of 49% aqueous hydrobromic acid solution (20 ml), and the mixture was heated at 120-125° C. The resulting mass was stirred for 2 hours at 120-125° C., and then the mass was cooled to 20-25° C. The resulting solution was decanted to another flask and the pH of the solution was adjusted to 11-12 with 50% aqueous sodium hydroxide solution (70 ml). The resulting aqueous layer was extracted two times with ethyl acetate (each time 50 ml). The total organic layer was washed with water (50 ml), dried over sodium sulfate, and then solvent was distilled completely under vacuum to give the product as an oily form. Isopropyl alcoholic HCl (3 ml) and ethyl acetate (20 ml) were added to the resulting oily mass, the solvents were distilled off completely, the resulting solid was co-distilled with methanol (50 ml), and then degassed for 1-2 hours at 70-75° C. The resulting solid was dissolved in methanol (25 ml) and methanol was distilled off completely to give an amorphous form of racemic rotigotine (HPLC Purity: 99.89%, Yield: 72%).

### Example 2

### Preparation of Amorphous Form of Racemic Rotigotine

Racemic rotigotine (2 gm) was dissolved in ethanol (20 ml). The solution was subjected to spray drying at about 65° C. for 6 hours to give 1.8 gm of an amorphous form of racemic rotigotine.

### Example 3

### Preparation of Crystalline Form of Racemic Rotigotine Free Base

Toluene (300 ml) was added to a round bottom flask, followed by the addition of aluminium chloride (43.5 g) and thiourea (18.6 g), and the resulting solution was stirred for 30 minutes at 20 to 25° C. This was followed by the addition of racemic 5-methoxy-2-[N-n-propyl-N-2-(2-thienyl)ethylamino]tetralin hydrochloride (30 g) in one lot and then stirred for 15 minutes at 20 to 25° C. The reaction mixture was heated at 110° C. for 1 to 2 hours. The resulting mass was allowed to cool at 0 to 5° C., water (140 ml) was added to the mass, followed by slow addition of aqueous ammonia (90 ml) and water (50 ml). This was followed by the addition of toluene (100 ml), stirred for 30 minutes and then filtered through cellite. The resulting organic layer was separated from the filtrate, washed with water (150 ml), and then dried over sodium sulfate and distillation of the toluene under vacuum at 50 to 55° C. to get oily mass. The resulting oil was co-distilled with n-heptane (50 ml) at 55 to 60° C., followed by the addition of n-heptane (200 ml), and then heating the mixture at 55 to 60° C. The resulting mass was gradually cooled to 0 to 5° C. under stirring. The resulting solid was filtered and washed with pre-cooled n-heptane (25 ml) and then dried for 12 hours at 30 to 35° C. to give 21.5 g of a crystalline form of racemic rotigotine free base.

Unless otherwise indicated, the following definitions are set forth to illustrate and define the meaning and scope of the various terms used to describe the invention herein.

The term “solid form of racemic rotigotine free base” includes crystalline forms, amorphous forms, hydrated forms, and solvated forms of racemic rotigotine.

The term “crystalline polymorph” means a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous forms of racemic rotigotine specifically contain less than about 10% crystalline forms of racemic rotigotine, more specifically less than 5% crystalline forms of racemic rotigotine, and still more specifically is essentially free of crystalline forms of racemic rotigotine. “Essentially free of crystalline forms of racemic rotigotine” means that other crystalline polymorph forms of racemic rotigotine not detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” means a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions described herein encompass compositions made by admixing the active ingredient, active ingredient dispersion or composite, additional active ingredient(s), and pharmaceutically acceptable excipients.

The expression “pharmaceutically acceptable salt” means those salts which are, within the scope of sound medical judgment, suitable for use in contact with the tissues of humans and lower animals without undue toxicity, irritation, allergic response and the like, commensurate with a reasonable benefit/risk ratio, and effective for their intended use.

The term “therapeutically effective amount” means the amount of a compound that, when administered to a mammal for treating a state, disorder or condition, is sufficient to effect such treatment. The “therapeutically effective amount” will vary depending on the compound, the disease and its severity, and the age, weight, physical condition and responsiveness of the mammal to be treated.

The term “delivering” means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. The term wt % refers to percent by weight. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A solid state form of racemic rotigotine free base.

2. The solid state form of racemic rotigotine free base of claim 1, characterized by data selected from the group consisting of:
i) a powder X-ray diffraction pattern substantially in accordance with FIG. 1;
ii) an IR spectrum substantially in accordance with FIG. 2;
iii) a powder X-ray diffraction pattern substantially in accordance with FIG. 3;
iv) a powder X-ray diffraction pattern having peaks at about 9.11, 11.47, 13.11, 16.63, 18.86, 19.41, 19.97, 20.82 and 22.32±0.2 degrees 2-theta substantially as depicted in FIG. 3; and
v) a powder X-ray diffraction pattern having peaks at about 13.92, 17.83, 23.70, 23.94, 25.49, 27.15 and 27.46±0.2 degrees 2-theta substantially as depicted in FIG. 3.

3. The solid state form of racemic rotigotine free base of claim 1, which is in a crystalline form or in an amorphous form, and further has a purity of about 98% to about 99.99% as measured by HPLC.

4. The solid form of racemic rotigotine free base of claim 3, which is in anhydrous form, solvent-free form, in a hydrate form, or in solvate form.

5. An amorphous form of racemic rotigotine free base, characterized by data selected from the group consisting of:
i) a powder X-ray diffraction pattern substantially in accordance with FIG. 1; and
ii) an IR spectrum substantially in accordance with FIG. 2.

6. A crystalline form of racemic rotigotine free base, characterized by data selected from the group consisting of:
i) a powder X-ray diffraction pattern substantially in accordance with FIG. 3;
ii) a powder X-ray diffraction pattern having peaks at about 9.11, 11.47, 13.11, 16.63, 18.86, 19.41, 19.97, 20.82 and 22.32±0.2 degrees 2-theta substantially as depicted in FIG. 3; and
iii) a powder X-ray diffraction pattern having peaks at about 13.92, 17.83, 23.70, 23.94, 25.49, 27.15 and 27.46±0.2 degrees 2-theta substantially as depicted in FIG. 3.

7. A process for the preparation of a solid state form of racemic rotigotine free base, comprising:
a) providing a solution or suspension of racemic rotigotine free base in a solvent;
b) optionally, filtering the solution or suspension to remove any extraneous matter; and
c) substantially removing the solvent from the solution or suspension to afford the solid state form of an amorphous form of racemic rotigotine free base; or
d) isolating from the solution or suspension the solid state form of a crystalline form of racemic rotigotine free base.

8. The process of claim 7, wherein the solvent in step-(a) is selected from the group consisting of alcohols, ketones, hydrocarbons, chlorinated hydrocarbons, esters, and mixtures thereof.

9. The process of claim 8, wherein the solvent is selected from the group consisting of methanol, ethanol, n-propanol, isopropyl alcohol, n-butanol, isobutanol, tert-butanol, amyl alcohol, hexanol, acetone, methyl ethyl ketone, methyl isobutyl ketone, methyl tert-butyl ketone, ethyl acetate, methyl acetate, isopropyl acetate, tert-butyl methyl acetate, ethyl formate, methylene chloride, ethylene dichloride, chloroform, carbon tetrachloride, n-pentane, n-hexane, n-heptane, cyclohexane, toluene, xylene, and mixtures thereof.

10. The process of claim 9, wherein the solvent is selected from the group consisting of methanol, ethanol, isopropyl alcohol, ethyl acetate, n-hexane, n-heptane, cyclohexane, toluene, and mixtures thereof.

11. The process of claim 7, wherein the solution obtained in step-(a) is further subjected to carbon treatment or silica gel treatment.

12. The process of claim 7, wherein the solution obtained in step-(a) is further stirred at a temperature of about 30° C. to the reflux temperature of the solvent used for at least 20 minutes.

13. The process of claim 7, wherein the removal of the solvent in step-(c) is accomplished by distillation or complete evaporation of the solvent, spray drying, vacuum drying, lyophilization or freeze drying, or a combination thereof.

14. The process of claim 7, wherein the isolation of crystalline form of racemic rotigotine free base in step-(d) is carried out by forcible or spontaneous crystallization.

15. The process of claim 14, wherein the forcible crystallization is initiated by cooling, seeding, partial removal of the solvent from the solution, by combining an anti-solvent with the solution, or a combination thereof.

16. The process of claim 15, wherein the crystallization is carried out by cooling the solution under stirring at a temperature of below 30° C. for at least 30 minutes.

17. The process of claim 16, wherein the crystallization is carried out by cooling the solution under stirring at a temperature of about 0° C. to about 30° C. from about 1 hour to about 20 hours.

18. The process of claim 7, wherein the crystalline form of racemic rotigotine free base obtained in step-(d) is recovered by filtration, filtration under vacuum, decantation, and centrifugation, or a combination thereof; followed by drying under vacuum or at atmospheric pressure, at a temperature of about 25° C. to about 70° C.

19. A process for the preparation of (−)-(S)-5-Hydroxy-2-[N-n-propyl-N-2-(2-thienyl)ethylamino]tetralin or a pharmaceutically acceptable salt thereof, comprising resolving the solid racemic rotigotine free base of claim 1 with a optically active resolving agent.

20. A pharmaceutical composition comprising solid racemic rotigotine free base of claim 1 and one or more pharmaceutically acceptable excipients.

21. The pharmaceutical composition of claim 20, wherein the pharmaceutical composition is a transdermal patch, an ointment, a gel, a cream, a paste, a spray, a film, a plaster, a poultice, or a cataplasm.

22. The pharmaceutical composition of claim 20, wherein the solid racemic rotigotine free base has a D₉₀ particle size of less than or equal to about 400 microns.

23. The pharmaceutical composition of claim 22, wherein the 90 volume-% of the particles (D₉₀) is less than or equal to about 15 microns.
